(19) United States
(12) Patent Application Publication
(10) Pub. No.: US 2020/0058387 A1
(43) Pub. Date: Feb. 20, 2020

(54) METHODS AND SYSTEMS FOR A COMPANION ROBOT

(71) Applicant: IKKIWORKS PTY LIMITED, Strathfield, New South Wales (AU)

(72) Inventors: Colin Douglas STAHEL, Eastwood, New South Wales (AU); Clive David MCFARLAND, Denistone East, New South Wales (AU); Seaton Drew MCKEON, Yerrinbool, New South Wales (AU)

(21) Appl. No.: 16/499,573

(22) PCT Filed: Mar. 29, 2018
(86) PCT No.: PCT/AU2018/050291

§ 371 Date: Sep. 30, 2019

(30) Foreign Application Priority Data
Mar. 31, 2017 (AU) 2017901192

(51) Int. Cl. G16H 20/30; G06K 7/10; G06K 19/07; G16H 20/10

(52) U.S. Cl. CPC G16H 20/30; G16H 20/10; G06K 19/0723; G06K 7/10366

(57) ABSTRACT

A system and method for functioning as a companion robot (11) to provide services to a user, collect data and information about the local environment to the robot and the user, and communicate with the user and to a remote processing means. A plurality of sensors are adapted to: (i) probe an environment in which the companion robot and user is disposed and generate data in respect of the environment; and sense information about the user and generate data containing the information. A communication means communicates: (i) sensorially perceptive information to the user; and (ii) data containing information to a remote processing means. A processing means: (i) receives data from the sensors; (ii) processes the data to extract information therefrom and perform prescribed functions with the information; and (iii) provides output information and data to the communication means.

### FIELD OF THE INVENTION

This invention relates to methods and systems for a companion robot. The invention has particular utility in the healthcare environment, especially for therapeutic and well-being applications.

These applications include paediatric oncology patients, and applications involving extended patient treatment, which may include but are not limited to burns, trauma, rehabilitation, neurological disorders and the elderly, involving telemedicine, dementia, isolation and other applications in the medical/health care area generally.

### BACKGROUND

Any discussion of the prior art throughout the specification should in no way be considered as an admission that such prior art is widely known or forms part of common general knowledge in the field.

Companion robots are a form of social robot that is an artificial intelligence system designed to interact with humans. These types of robots have become popular as a result of technological advancements in artificial intelligence systems that allow the robot to be autonomous and have the ability to interact independently in response to cues from people and actions or events in the environment of the robot. Hence the robot can act as a companion to a human at home or in a healthcare facility.

Having this type of autonomous ability distinguishes companion robots from other types of robots such as industrial robots. Companion robots are often referred to as ‘smart’ or ‘intelligent’ robots. Smart robot intelligence is typically based on a cognitive computer model to stimulate human thought processes. Cognitive computing involves machine-learning systems that use data mining, pattern recognition and natural language processing to mimic the way the human brain works. This allows social robots to interact in increasingly sophisticated ways.

The healthcare environment provides an excellent application for companion robots to be developed that have the ability to accompany a patient, act as its friend, be a means of assistance, and provide a reassuring presence in troubling times.

Interaction with humans that are unwell and/or require therapeutic treatment provides a whole raft of complexities and problems that need to be addressed and overcome in the design of a companion robot in order for it to meet industry standards in the healthcare environment.

Current companion/therapy robots tend to fall into one of two types: (i) custom built, or (ii) off-the-shelf.

Custom-built robots (such as, MIT's Huggable™) have an advantage that they are specifically designed for the intended application. However, because they use custom hardware and software they tend to be complicated to design and manufacture and are typically expensive.

Off-the-shelf robots (such as, RxRobots™) are less complex to design and manufacture, but are not specifically designed for the intended application. Their modification for clinical use is therefore based on customised software. Adding any application-specific hardware, for example sensors such as temperature, is extremely difficult to do as the robots were not originally designed with this in mind.

Both custom and off the shelf robots have a range of mechanical components such as motors, gears and joints which are prone to failure. Taken together, these factors tend to make the conventional robots expensive and fragile.

Embodiments of the present invention seek to differentiate themselves from the prior art by being low cost, customisable, and robust with a minimum of mechanical components.

It is an object of the present invention to overcome or ameliorate at least one of the disadvantages of the prior art, or to provide a useful alternative.

### SUMMARY OF THE INVENTION

Advantageously, preferred embodiments of the present invention provide methods and systems to enable a companion robot to perform specified functions to an acceptable standard prescribed by the environment in which it is intended to operate. These functions may include, for example, providing services to a person, collecting data and information about the environment local to the robot and/or about the person, and communicating with the person and/or to a processing means

In accordance with a first aspect of the present invention, there is provided a system for functioning as a companion robot for a user, the system including: one or more sensors adapted to:

(i) probe an environment in which the companion robot and user is disposed and generate data in respect of the environment; and

(ii) sense information about the user and generate data containing the information;
communication means to communicate:

(i) sensorially perceptive information to the user; and

(ii) data containing information to a remote processing means;
processing means to:

(i) receive data from each sensor;

(ii) process the data to extract information therefrom and perform predetermined functions using the information; and

(iii) provide output information and data to the communication means to communicate; and
an external casing at least substantially encapsulating the system.

Preferably, the sensors include one or more of the following:
(i) a distance sensor for determining the distance between the surface of an object in a line of sight and the location of the distance sensor;
(ii) a temperature sensor for determining the ambient temperature around an external casing of the system;
(iii) a motion sensor;
(iv) an RFID (radio frequency identification) interrogation sensor for detecting and interrogating a remote RFID tag associated with the system when the tag is brought into a predetermined proximity to the RFID interrogation sensor;
(v) a tactile sensor for determining when the casing is touched in a predetermined manner by an external object.

Preferably, the motion sensor includes one or more of an accelerometer, a speedometer, a gyroscope, and a global positioning system (GPS).

Preferably, the processing means includes one or more of the following processes to perform one or more of the predetermined functions:
(i) an initialisation process invoked for coupling the system to a user;
(ii) a recharging process invoked for charging a battery resident with the system for delivering locally supplied power to the system;
(iii) an RFID interrogating process invoked by the RFID interrogation sensor for identifying an object bearing the remote RFID tag and performing a prescribed processing function in relation to the object.

Preferably, the initialisation process includes a triggerable voice recognition system that is primed to recognise, respond and adapt to the voice of the user. Preferably, the recharging process is triggerable when the system is connected to a remote power supply.

Preferably, the communication means includes one or more of the following communication mechanisms:
(i) lighting;
(ii) sound;
(iii) movement and
(iv) wireless;
wherein each of the communication mechanisms are individually triggerable by a related process of the processing means.

Preferably, the lighting communication mechanism includes at least one light source disposed to illuminate different parts of the casing. The light source is preferably selected from the group including: LEDs and arrays thereof, LCD screens and matrices, OLED screens and matrixes, lamps. Preferably, the sound communication mechanism includes multiple synthetic and recorded sounds. Preferably, the movement communication mechanism includes a vibration generator or motor. Preferably, the wireless communication mechanism is able to communicate data and information collected by the system to a smart client device, and to receive data and information for the system from the smart client device.

Preferably, the processing means includes a medication compliance process. Preferably, the medication compliance process includes one or more of the following sub-processes which are performed in respect of an associated medication event:
(i) a medication reminder invoking process;
(ii) a medication verification process;
(iii) a correct medication asserting process; and
(iv) a logging process for logging the medication event.

Preferably, the medication reminder invoking process triggers a reminder process for reminding the user or patient to take medication at prescribed times as derived from a medication schedule. The medication reminder invoking process preferably includes a medication schedule interrogation process for interrogating the medication and extracting the timing data stored in the medication schedule to trigger. Preferably, the medication schedule includes prescribed medicines for the patient to take, the container identity for containing the prescribed medicine, and the scheduled times for these to be taken.

Preferably, the medication verification process includes identifying the correct medication by verifying the identity of a container containing the medication disposed within the prescribed proximity to the RFID interrogation sensor. Preferably, the container includes an RFID tag including the container identity.

Preferably, the correct medication asserting process includes generating a sensorially perceptible signal to the communication means. The signal preferably either validates the selection of the container when its sensed identity corresponds with the medication prescribed for the container according to the medication schedule, or invalidates the selection of the container when its sensed identity does not correspond with the medication prescribed for the container according to the medication schedule.

Preferably, the processing means includes a temperature taking process. The temperature taking process is preferably programmed to perform one or more of the following sub-processes in respect of a temperature taking event:
(i) a temperature reminder invoking process;
(ii) a temperature calibration process;
(iii) a temperature orientation process to direct the user to orient the system into the correct orientation for the temperature measurement;
(iv) a temperature positioning process to direct the user to position the system at the correct distance from the user's skin for optimal measurement accuracy;
(v) a temperature reading process;
(vi) a temperature comparison process;
(vii) a temperature alert process; and
(viii) a logging process for logging the temperature taking event.

Preferably, the temperature reminder invoking process triggers a reminder process for reminding the user to take their temperature at prescribed times as derived from a temperature schedule. Preferably, the reminder process uses specific audio-visual cues generated by the corresponding communication mechanism.

Preferably, the temperature calibration process includes checking the infra-red temperature sensor to ensure the accuracy of the temperature measurement. Preferably, if the check returns a failed result, specific audio-visual cues are generated by the corresponding communication mechanism, the failure is logged, and the temperature measurement does not proceed.

Preferably, temperature orientation process includes providing orientation cues to the user. The orientation cues are preferably audio and/or visual cues. The orientation cues preferably direct the user to use both hands to pick the system up by the wings with the beak facing the user such that the system is in the correct orientation for the temperature measurement.

Preferably, the temperature positioning process includes providing visual cues to the user. The visual cues preferably direct the user to place the system against the user's forehead such that the infra-red sensor is automatically positioned at the correct distance from the skin for optimal measurement accuracy.

Preferably, the temperature reading process includes initiating a temperature reading and continually analysing the data so obtained via an appropriate methodology until the analytical method indicates that a stable reading has been obtained. The methodology may include, for example, fixed average, moving average, or linear regression techniques.

Preferably, the temperature comparison process includes comparing the temperature reading with stored set points indicating low, normal and high temperatures, and generating appropriate audio-visual cues via the corresponding communication mechanism.

Preferably, the temperature alert process includes, when appropriate, providing alerts to parents, guardians or clinical staff via mechanisms such as Bluetooth, WiFi, GSM or SMS.

Preferably, the logging process for logging events includes:
(i) a missing event tracking process for logging missed events;
(ii) an alert function for invoking the communication means to send an alert signal to the remote processing means in accordance with an alert profile customised for the user in respect of events that have been missed for a prescribed number of times; and
(iii) an alert profile interrogating process to derive an alert flag for missing events from the alert profile and trigger the alert function when the missed number of events tracked by the missing event tracking process matches the alert flag.

Preferably, the logging process can be used for logging the medication event, and the events are medical events. Preferably, the logging process can be used for logging the temperature taking event, and the events are temperature taking events. The logging process preferably includes logging any one or more data items created during the event. Preferably, logged data items are subsequently made available for review by a clinical expert. For a medication event, the logging process may include logging data items related to if the medication was correct, if the medication was incorrect, and/or the time medication was taken. For a temperature taking event, the logging process may include logging data items related to the time temperature was taken, and/or the temperature reading.

Preferably, the processing means includes a frustration process that is programmed to perform the following functions in respect of a frustration event:
(i) reading the accelerometer repeatedly once the frustration process has initiated;
(ii) determining if a change of greater than a specified threshold has been detected in the x, y or z axis of movement between two successive readings; and
(iii) if such a change is detected, providing a cue to the user.

Preferably, the accelerometer is read approximately every 100 milliseconds. Preferably the specified threshold is about 0.3 units. Preferably, the cue includes a visual cue and/or an audible cue. Preferably, the visual cue is a light pulse of a prescribed duration. Preferably, the prescribed duration is about 0.5 seconds. Preferably, the audible cue is in the form of a brief sound selected at random from a library of such sounds.

Preferably, the processing means includes a reminder process. The reminder process preferably includes:
(i) an audio-visual cue selector to invoke the communication means and generate a corresponding communication mechanism related to the particular type of invoking process triggering the reminder process;
(ii) a time reminder to signal the communication means to assert sensorially perceptible information indicative of the event being reminded in the absence of a response within a prescribed time from the initial reminder;
(iii) a medication reminder or temperature taking reminder to signal the communication means to assert sensorially perceptible information indicative of a medication reminder or temperature taking reminder, as appropriate.

Preferably, the processing means invokes a calming process. The calming process is preferably programmed to perform one or more of the following sub-processes in respect of a calming event:
(i) a light cue process;
(ii) an audio cue process;
(iii) a temperature reading process;
(iv) a high temperature detection process;
(v) a breathing detection process.

Preferably, the light cue process includes illuminating a light source in a region of the system for a prescribed period and instructing the user to blow towards the region. Preferably, the light source is illuminated a predetermined colour. In one example, the colour is orange. Preferably the prescribed period is about 2 seconds. Preferably, the user is instructed by an audible cue to ‘blow out the candles’.

Preferably, the temperature reading process includes initiating a temperature reading on a substantially continuous periodic basis and taking the moving average of a prescribed number of consecutive readings. Preferably, the temperature reading occurs every 20 milliseconds. Preferably, the prescribed number of consecutive readings is 30.

Preferably, the high temperature detection process includes determining if the average temperature reading has remained high for a predetermined number of consecutive readings and if so, providing at least one cue. Preferably, the cue includes a visual cue in the form of extinguishing the light source. Preferably, the cue includes an audible cue to denote that a breath has been detected. Preferably, the predetermined number of consecutive readings is 100.

Preferably, the breathing detection process includes determining if a breathing event has occurred. Preferably, if no breathing event is detected, the light source is extinguished for a predetermined period before the cycle is repeated. Preferably, if a breathing event is detected, the light source is extinguished for time period increased by a prescribed factor and the cycle is repeated. Preferably, the prescribed factor is 1.5. Preferably, if several consecutive breathing events are detected such that the ‘off’ period of the cycle reaches a preset value, the length of this period remains at this preset value and is not further increased.

Preferably, the processing means invokes a voice sensing and modifying process to sense when a vocal sound is generated by a user, including:

a linguistic detection process; and

a voice responding process for generating a response based on the sensed vocal sound.

Preferably, the voice responding process includes:
(i) use of a microphone to detect vocal sounds;
(ii) conversion of the analogue audio signal generated by the microphone into a digital format;
(iii) use of real-time sound processing software to modify the digital audio signal as it is received;
(iv) use of audio components to play back the modified audio signal.

In another embodiment, the voice responding process includes analysis of a detected vocal sound, and generation of a response relevant to the result of the analysis.

Preferably, the processing means includes a motion process, including:
(i) use of the motion sensor to measure movement in the x, y and z planes of movement;
(ii) analysis of the measured movement; and
(iii) generation of an appropriate response.

One preferred example of the motion process includes the detection of a motion signature characteristic of a gentle lateral rocking motion and the generation of audio-visual responses characteristic of sleeping.

Preferably, the processing means includes one or more of a tactile process for interacting with the tactile sensors, a game loader, a lighting function, and various other functions capable of being implemented on a hardware and software platform using artificial intelligence and communicating across various media including the Internet.

Preferably, the system includes data storage including a sensed data storage area for storing sensed data derived from the logging process logging a medication event or a temperature-taking event. Preferably, the sensed data is composed for transmission or upload by the communication means in response to the processing means receiving a transmission signal from the communication means. Preferably, the storage and transmission of data will be consistent with industry standard security protocols, such as the use of encryption algorithms for secure storage and transmission.

Preferably, the data storage includes a template storage area for storing one or more of the medication schedule, the temperature schedule, the breathing template, and other schedules and templates that may be personalised to the user.

Preferably, the external casing includes a protrusion and a confronting zone disposed a fixed distance apart for the purposes of targeting temperature taking.

In accordance with another aspect of the present invention, there is provided a method for functioning as a companion robot for a user, the method including:
(i) probing an environment in which the companion robot and user is disposed and generating data in respect of the environment;
(ii) sensing information about the user and generating data containing the information;
(iii) communicating sensorially perceptive information to the user and data containing information to a processing means; and
(iv) performing prescribed functions with the data in respect of the environment and the information about the user.

Further features and advantages of the present invention will become apparent to those of ordinary skill in the art in view of the detailed description of preferred embodiments below.

### BRIEF DESCRIPTION OF THE DRAWINGS

A preferred embodiment of the invention will now be described, by way of example only, with reference to the accompanying drawings in which:

FIG. 1 is a front perspective view of a companion robot according to an embodiment of the present invention;

FIG. 2 is a rear perspective view of the companion robot shown in FIG. 1;

FIG. 3 is a front view of the companion robot shown in FIG. 1;

FIG. 4 is a left side view of the companion robot shown in FIG. 1;

FIG. 5 is a rear view of the companion robot shown in FIG. 1;

FIG. 6 is a right side view of the companion robot shown in FIG. 1;

FIG. 7 is a top view of the companion robot shown in FIG. 1;

FIG. 8 is a bottom view of the companion robot shown in FIG. 1;

FIG. 9 is an exploded perspective view of the companion robot shown in FIG. 1 and a charging dock for same, showing the principal components making up the companion robot and charging dock;

FIG. 10 is a schematic block diagram showing the hardware architecture for the companion robot of FIG. 1;

FIG. 11 is a diagram showing the core principles of the companion robot shown in FIG. 1;

FIG. 12 is a diagram showing an example customisation for a specific application relating to the core principles shown in FIG. 11;

FIG. 13 is a flow chart showing the start-up routine and main control loop that invokes preferred principal functions performed by the companion robot shown in FIG. 1;

FIG. 14 is a sequence flowchart showing the processes performed for implementing an optional ‘calming and breathing’ function of the companion robot shown in FIG. 1;

FIG. 15 is a sequence flowchart showing the processes performed for implementing an optional ‘temperature measurement’ function of the companion robot shown in FIG. 1;

FIG. 16 is a sequence flowchart showing the processes performed for implementing an optional ‘medication compliance’ function of the companion robot shown in FIG. 1;

FIG. 17 is a sequence flowchart showing the processes performed for implementing an optional ‘night light’ function of the companion robot shown in FIG. 1;

FIG. 18 is a sequence flowchart showing the processes performed for implementing an optional ‘sounds and stories’ function of the companion robot shown in FIG. 1;

FIG. 19 is a sequence flowchart showing the processes performed for implementing an optional ‘babble speak’ function of the companion robot shown in FIG. 1;

FIG. 20 is a sequence flowchart showing the processes performed for implementing an optional ‘ouch response’ function of the companion robot shown in FIG. 1;

FIG. 21 is a sequence flowchart showing the processes performed for implementing an optional ‘wake up sequence’ function of the companion robot shown in FIG. 1;

FIG. 22 is a flowchart showing how the temperature measurement function performs when invoked from the main control loop;

FIG. 23 is a flowchart showing how the nightlight/update function performs when invoked from the main control loop; and

FIG. 24 is a flowchart showing how the switch polling is performed as part of the main control loop.

### DESCRIPTION OF THE PREFERRED EMBODIMENTS OF THE INVENTION

Preferred embodiments of the invention are directed towards a companion robot functioning to provide services to a user, collect data and information about the local environment to the robot and the user, and communicate with the user and to a remote processing means.

Here, the user may be a child or other patient undergoing extended therapy and treatment. The user may be in a number of different environments including an environment that is well supported with healthcare services and medical personnel, such as a hospital, and an environment that is not so well supported with healthcare services and medical personnel, such as the home. One example may be ‘oncology’ applications. However, it will be appreciated that the invention will be applicable to a wide range of users, environments and alternative applications.

A preferred embodiment of invention characterises the companion robot 11 in the form of a baby penguin to appeal particularly to children undergoing treatment. It will be appreciated that the companion robot may take any form. Additionally, a number of different external casings or casing components may be available, so that the user can customise the appearance of their companion robot.

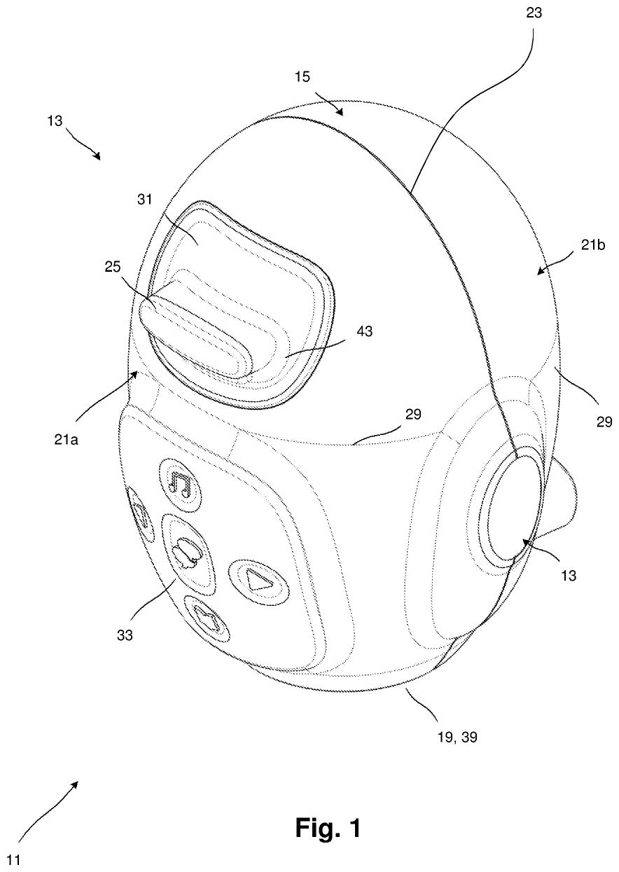

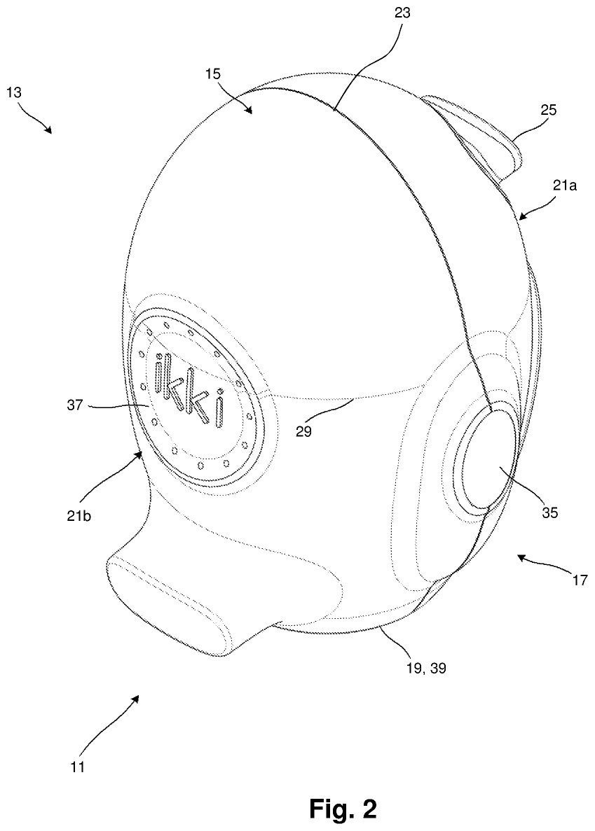

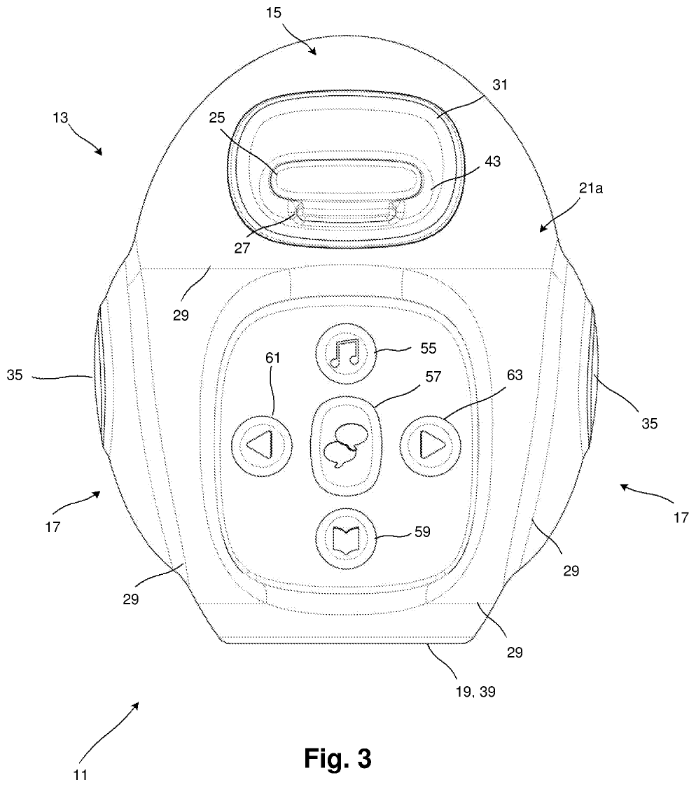

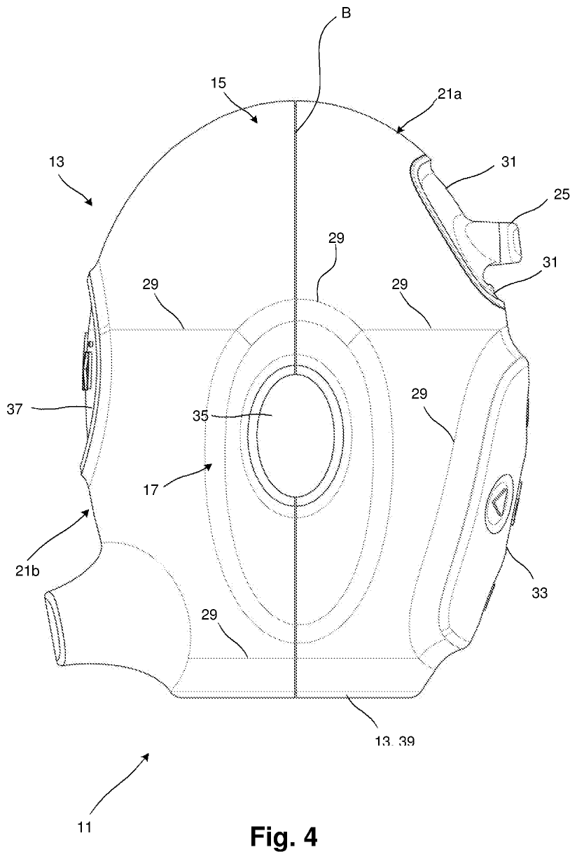

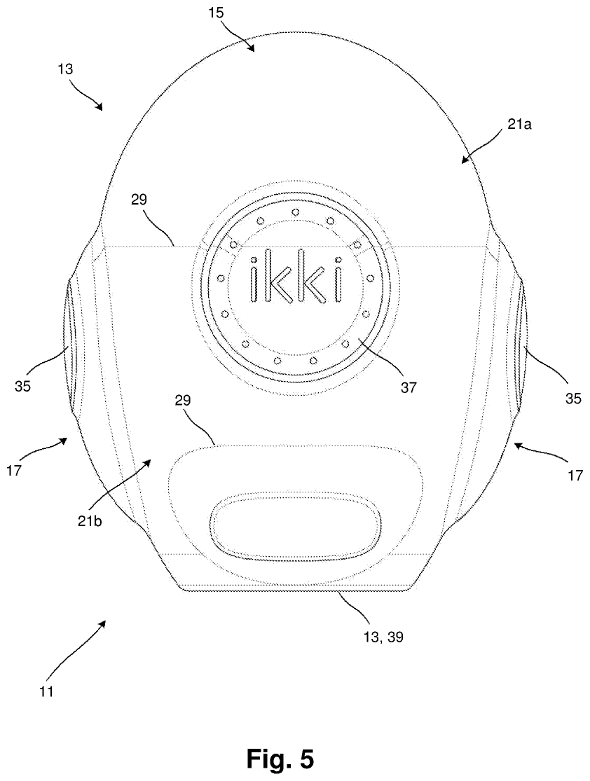

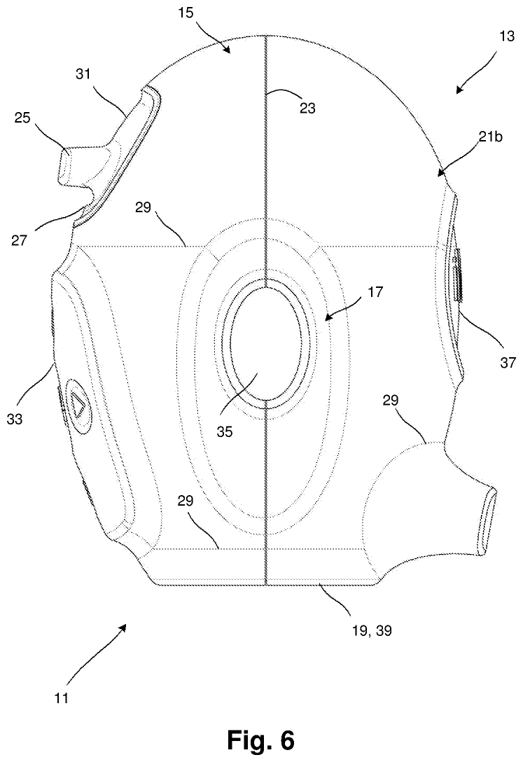

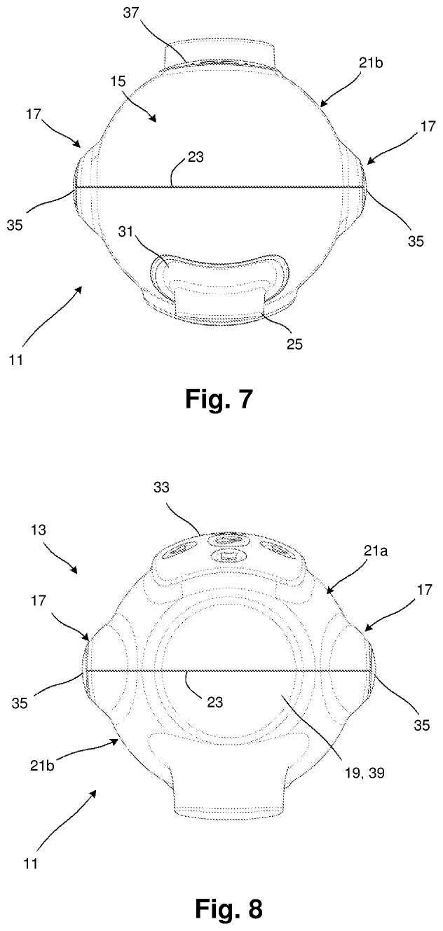

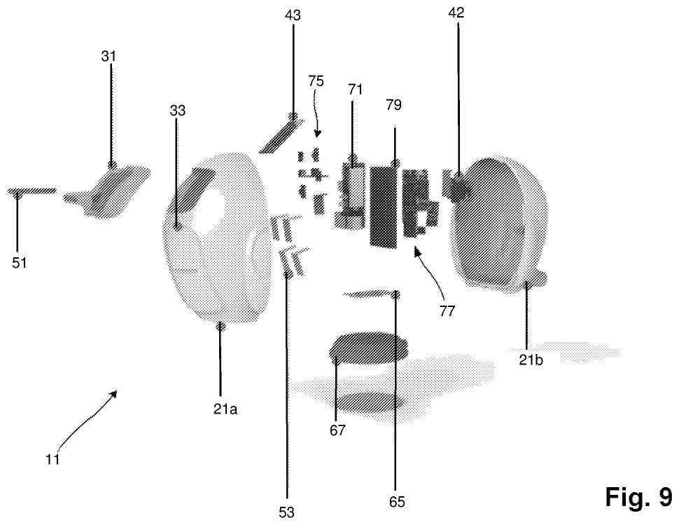

By adopting the baby penguin form, as shown in FIGS. 1 to 9, the robot 11 includes a substantially ovoid body 13 having an integral head portion 15 simulating the head of the penguin, expanded sides 17 simulating the wings of the penguin, a flat base 19 simulating the feet of the penguin on which the robot may stand upright, and a front chassis 21a and a rear chassis 21b simulating the front and sides of the penguin.

The head portion 13 includes a protrusion 25, simulating the beak of the baby penguin. The head portion 13 also includes a recess 27 disposed directly beneath the protrusion 25, simulating the mouth of the baby penguin.

The front and rear chassis 21 effectively comprise two halves of a shell that are lockingly clipped together along a circumferential planar centre line 23 that extends through the expanded sides 17 and the head and base portions 15,19 to form an external casing of the body 13 housing various internal components of the robot 11.

Each chassis 21 incorporates a plurality of demarcated skins on the surface thereof made of flexible material, the skins spanning prescribed demarcated regions of the body bounded within score lines 29 and prescribed components to provide a degree of resilience and responsiveness to tactile forces arising from handling of the robot.

The prescribed components specifically include a light diffuser faceplate 31, a button membrane 33, control pads 35, a rear label pad and speaker 37, and a charging base 39 including a speaker panel 41 for a speaker 42 disposed within the chassis 21. The control pads 35 are preferably capacitive touch switches which can be programmed for a variety of different purposes. In the described embodiment, the control pads are used for volume control and for temperature measurement. For volume control, the pads may be touched to move the desired volume up or down. For temperature measurement, the area around the pad illuminates to initiate the temperature measurement reading. The control pads then detect when they are both being touched simultaneously, that is, when the device is being held in the correct orientation, and a temperature measurement is then taken. The control pads 35 may also provide other visual cues to the user for positioning and orienting the robot.

The faceplate 31 includes an infrared sensor 43, along with: (i) the protrusion 25 incorporating an RFID (radio frequency identification) sensor 45; and (ii) the recess 27 incorporating an RFID tag slot 47. The RFID tag slot 47 is particularly sized and designed to receive a data or information carrier 49, including a RFID tag 51 for selective insertion into the slot 47.

The button membrane 33 includes a plurality of input buttons that operate corresponding switches 53 and include a music function button 55, a babble/record button 57, a story function button 59, a library back button 61 and a library skip button 63.

The charging base 39 includes a contact panel (not shown) to contact a charging module 65 forming part of a charging dock 67. The charging module may be an inductive charger or a mechanical connection such as a plug.

The chassis 21 of the companion robot 11 also includes processing means including a central processing unit (CPU) 71 that interfaces with the various sensors and buttons as previously described using physical interface circuitry componentry provided on a printed circuit board mounting the CPU and related componentry 75.

A communication means including a communication processing circuit 77 is connected to: the various input and output components, including the sensors, buttons and speaker 42 previously described; and the CPU 71.

The various circuitry including the CPU 71, communication processing circuit 77 and appropriate peripheral componentry are powered by a battery 79.

The various components described form a system for functioning as a companion robot 11 to provide services to a user, being a patient having a medical condition in the present embodiment, and medical personnel associated with treating the patient in respect of that medical condition. In a preferred embodiment, the patient is a child, but may be a patient of any age. The companion robot 11 in this manner functions to collect data and information about the environment local to the robot and the patient, and communicates with the patient in a manner that appeals to and incites interaction with the patient, and communicates with a remote processing means supporting the robot.

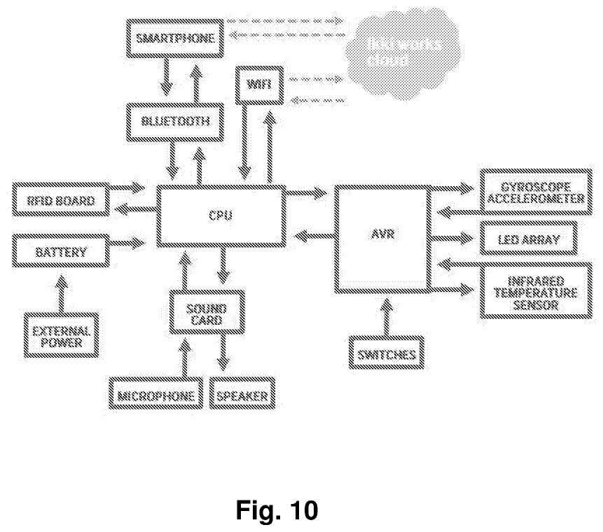

To achieve this end, the system includes a hardware architecture as shown in FIG. 10.

The peripheral components that form part of the system in the present embodiment are arranged to include a plurality of sensors adapted to probe the environment in which the companion robot and patient is disposed and generate data in respect of the environment. They are also adapted to sense information about the patient and generate data containing the information.

The communication means is adapted to communicate various information and data including: sensorially perceptive information to the patient; and data containing information to the remote processing means.

The processing means is adapted to:
(i) receive data from the sensors;
(ii) process the data to extract information therefrom and perform prescribed functions with the information; and
(iii) provide output information and data to the communication means to communicate.

The sensors take on various forms. These may include a distance sensor for determining the distance between the surface of an object in a line of sight and the location of the distance sensor relative to the system.

A temperature sensor may be included for determining the ambient temperature around an external casing of the system.

A motion sensor may be included. The motion sensor may include any one or more of an accelerometer, a speedometer, a gyroscope, and a global position system (GPS).

The RFID sensor 43 is an interrogation sensor for detecting and interrogating a remote RFID tag 51 associated with the system when brought into a prescribed proximity to the RFID interrogation sensor 43 by inserting it into the slot.

The skins overlie tactile sensors for determining when the casing 21 is touched in a prescribed manner by an external object. These are interfaced with the CPU 71 by appropriate peripheral componentry, to all the processing means including the CPU to perform various specified functions. These functions are performed by software processes including:
(i) an initialisation process invoked for coupling the system to a user, the initialisation process including a triggerable voice recognition system that is primed to recognise, respond and adapt to the voice of the user;
(ii) a recharging process invoked for charging a battery resident with the system for delivering locally supplied power to the system, the recharging process being triggerable when the system is connected to a remote power supply; and
(iii) an RFID interrogating process invoked by the RFID interrogation sensor for identifying an object bearing a remote RFID tag and performing a prescribed processing function in relation to the object.

The communication means includes various communication mechanisms including:
(i) lighting including a light disposed to illuminate different parts of the casing, the light source including one or more of LEDs and arrays thereof, LCD screen and matrices, OLED screens and matrices, and lamps;
(ii) sound including multiple synthetic and recorded sounds;
(iii) movement including a vibration generator or motor; and
(iv) wireless to communicate data and information collected by the system to a smart client device in relatively close proximity to the system, and to receive data and information for the system from the smart client device.

Each of the communication mechanisms are individually triggerable by a related process of the processing means.

The processing means includes a medication compliance process including sub-processes programmed to perform the prescribed functions in respect of a medication event. These sub-processes include:
(i) a medication reminder invoking process to trigger a reminder process for reminding the user to take medication at prescribed times as derived from a medication schedule, the medication reminder invoking process including a medication schedule interrogation process for interrogating the medication and extracting the timing data stored in the medication schedule to trigger;
(ii) a medication verification function for identifying the correct medication by verifying the identity of a container containing the medication disposed within the prescribed proximity to the RFID interrogation sensor, the container bearing an RFID tag including the container identity;
(iii) a correct medication asserting function for generating a sensorially perceptible signal to the communication means either validating the selection of the container when its sensed identity corresponds with the medication prescribed for the container according to the medication schedule, or invalidating the selection of the container when its sensed identity does not correspond with the medication prescribed for the container according to the medication schedule; and
(iv) invoking a logging process for logging the medication event.

The medication schedule includes prescribed medicines for the user to take, the container identity for containing the prescribed medicine, and the scheduled times for these to be taken.

The processing means includes a temperature taking process that is implemented by way of a temperature check function which is one of the primary features distinguishing the companion robot 11 from other toys on the market. It includes an interface with a smartphone app to enable it to communicate temperature data with the remote processing means interfaced to a hospital network. The process is programmed to perform functions in respect of a temperature-taking event. The functions include:
(i) a temperature reminder invoking process to trigger the reminder process for reminding the user to take their temperature at prescribed times as derived from a temperature schedule using specific audio-visual cues generated by the corresponding communication mechanism;
(ii) initiating a calibration event whereby the infra-red temperature sensor is checked to ensure the accuracy of the temperature measurement, whereby if the calibration test fails, specific audio-visual cues are generated by the corresponding communication mechanism, the failure is logged, and the temperature measurement does not proceed;
(iii) providing audio-visual cues for the user to use both hands to pick the robot up by the wings with the beak facing the user such that the robot is in the correct orientation for the temperature measurement;
(iv) providing visual cues to place the beak of the robot against the user's forehead such that the infra-red sensor is automatically positioned at the correct distance from the skin for optimal measurement accuracy;
(v) initiating a temperature reading and continually analysing the data so obtained via an appropriate methodology (for example fixed average, moving average, linear regression) until the analytical method indicates that a stable reading has been obtained;
(vi) the temperature reading with stored set points indicating low, normal and high temperatures, and generating appropriate audio-visual cues via the corresponding communication mechanism;
(vii) when appropriate, providing alerts to parents, guardians or clinical staff via mechanisms such as Bluetooth, WiFi, GSM or SMS;
(viii) invoking the logging event for logging the temperature taking event.

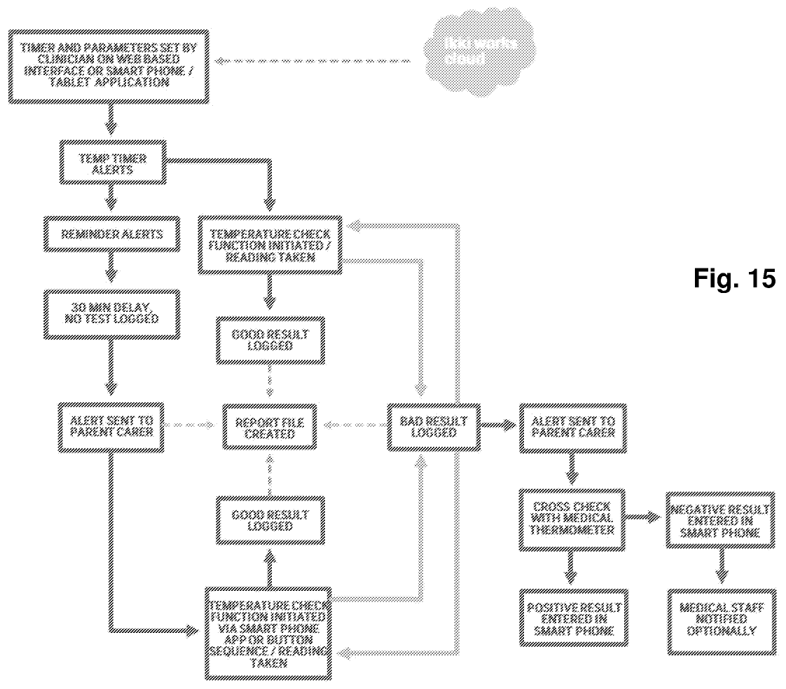

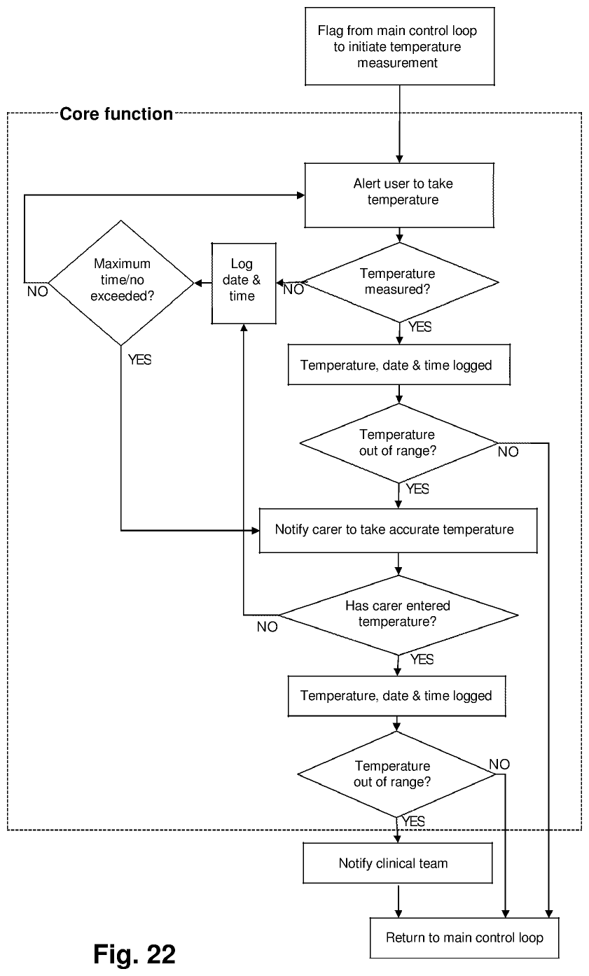

The temperature check function is implemented in software according to the flowchart shown in FIG. 15. A detailed temperature measurement function is provided by the temperature check function when invoked from the main control loop is implemented according to the flowchart shown in FIG. 22.

The logging process is implemented by way of a medication compliance function that provides real world data logs of medication for the clinicians to see what is happening outside of the hospital to better gauge and understand patient progress and status. It includes:
(i) a missing event tracking process for logging missed medication events or temperature taking events;
(ii) an alert function for invoking the communication means to send an alert signal to the remote processing means in accordance with an alert profile customised for the user in respect of medication events or temperature taking events that have been missed for a prescribed number of times; and
(iii) an alert profile interrogating process to derive an alert flag for missing events from the alert profile and trigger the alert function when the missed number of events tracked by the missing event tracking process matches the alert flag.

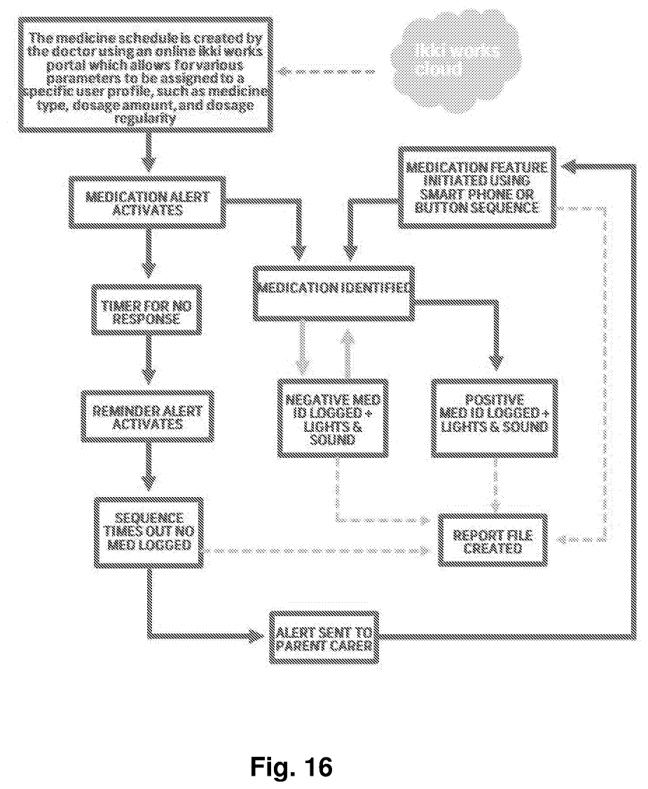

The medication compliance process is implemented in software according to the flowchart shown in FIG. 16.

The processing means includes a frustration process that is programmed to perform functions in respect of a frustration event. In the present embodiment, the frustration process is referred to as an ‘ouch’ response, which is a simple feature that normalises the suffering endured by the user and allows them to inflict some ‘pain’ onto the companion robot, as they are often frustrated and powerless when undergoing various surgeries and medical procedures.

The functions performed by the ouch response are as follows:
(i) reading the accelerometer every 100 milliseconds once the frustration process has initiated;
(ii) determining if a change of above a specified threshold of about 0.3 units has been detected in the x, y or z axis of movement between two successive readings; and
(iii) if such a change is detected, providing a visual cue in the form of a light pulse of a prescribed duration of 0.5 seconds, and an audible cue in the form of a brief sound selected at random from a library of such sounds.

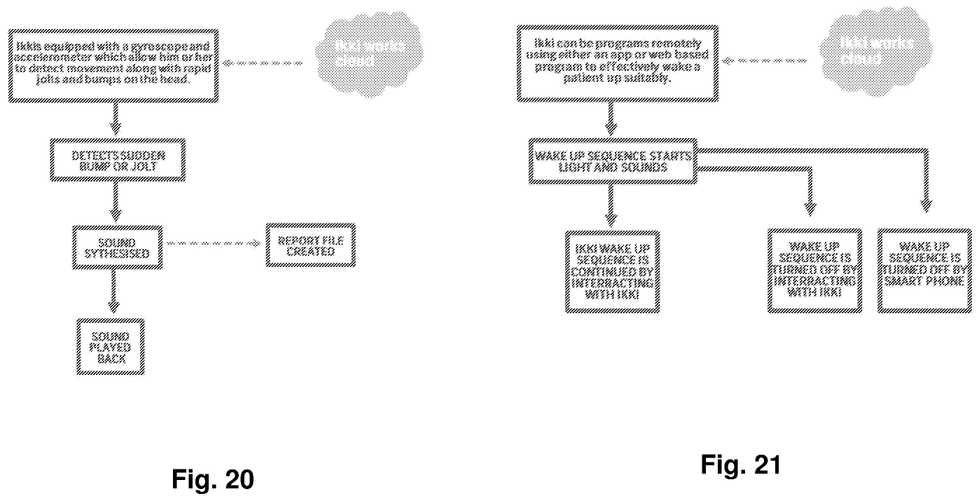

The ouch response in the present embodiment is implemented in software according to the flowchart shown in FIG. 20.

The processing means also includes a reminder process, which includes:
(i) an audio-visual cue selector to invoke the communication means and generate a corresponding communication mechanism related to the particular type of invoking process triggering the reminder process;
(ii) an appealing time reminder to signal the communication means to assert sensorially perceptible information indicative of the event being reminded in the absence of a response within a prescribed time from the initial reminder; and
(iii) an appealing medication reminder or temperature taking reminder to signal the communication means to assert sensorially perceptible information indicative of a medication reminder or temperature taking reminder, as appropriate.

The processing means also invokes a calming process known as a calming and breathing function that encourages the patient to focus on their breathing in order to relax and calm down. The function may be used to help in stressful situations or for remedial therapies, for example wellbeing and mindfulness, through its ability to adapt and extend to the breathing capacity of the user. Essentially it is programmed to perform the following functions in respect of a calming event:
(i) initiating a light cue process whereby the lights in the face region are illuminated a prescribed colour, being orange in the present embodiment, for a corresponding prescribed period, of for example 2 seconds, and the user is instructed to incite blowing into the face region of the robot by, for example, the robot saying ‘blow out the candles’;
(ii) initiating a temperature reading on a substantially continuous periodic basis of every 20 milliseconds and taking the moving average of a prescribed number of consecutive readings, being 30 in the present embodiment;
(iii) determining if the average temperature reading has remained high for a predetermined number of consecutive readings being 100 in the present embodiment, and if so, providing a visual cue by turning the prescribed colour (for example orange) face lights off and an audible cue to denote that a breath has been detected;
(iv) if no breathing event is detected the prescribed colour orange face lights are extinguished for a corresponding period of for example 2 seconds and the cycle is repeated;
(v) if a breathing event is detected, then the length of time the prescribed colour orange face lights remain off for is increased by a prescribed factor of for example 1.5 and the cycle is repeated;

(vi) if several consecutive breathing events are detected such that the ‘off’ period of the cycle reaches a preset value, the length of this period remains at this preset value and is not further increased.

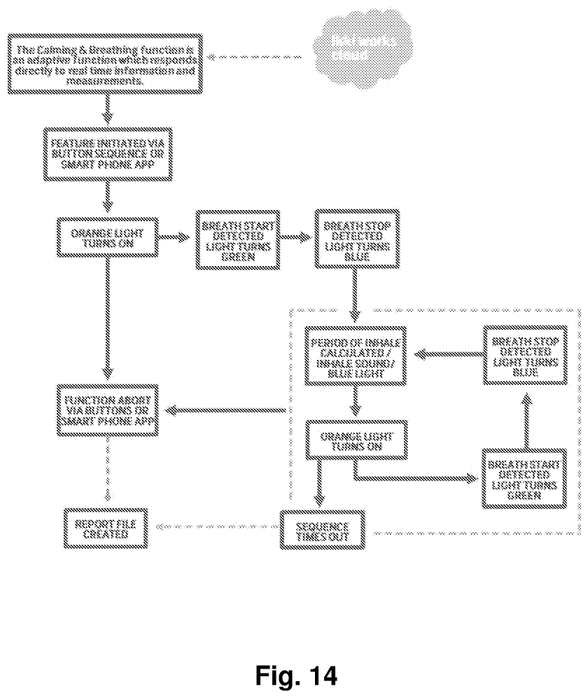

A flowchart showing how the software process for the calming and breathing function is implemented is shown in FIG. 14

The processing means invokes a voice sensing and modifying process to sense when a word or phrase is spoken by the user, including: a linguistic detection process; and a voice responding process for generating a response based on the vocal input supplied.

The voice responding process makes use of a microphone to detect vocal sounds and an appropriate system to convert the analogue audio signal generated by the microphone into a digital format.

It also makes use of real-time sound processing software to modify the digital audio signal as it is received; and use of an appropriate system such as a soundcard, audio amplifier and loudspeaker to play back the modified audio signal.

The processing means includes a motion process. The motion process includes:
(i) use of the motion sensor to measure movement in the x, y and z planes of movement;
(ii) analysis of the measured movement; and
(iii) generation of an appropriate response.

One example is the detection of a motion signature characteristic of a gentle lateral rocking motion and the generation of audio-visual responses characteristic of sleeping. Another example is the detection of the robot being lifted or moved quickly and the robot responding with a ‘whooshing’ sound. The motion process may be used to implement the ‘ouch’ response, using an accelerometer to detect sudden movements or forces applied to the robot. Further examples may include the use of a speedometer and/or GPS device for a racing game, use of a GPS for hide and seek, and use of a gyroscope for an orientation-type game.

The processing means also includes a tactile process for interacting with the tactile sensors, a game loader, a lighting function and various other functions capable of being implemented on a hardware and software platform using artificial intelligence and communicating across various media including the Internet.

The system includes secure data storage including a sensed data storage area for storing sensed data derived from the logging process logging a medication event or a temperature-taking event, whereby the sensed data is composed for transmission or upload by the communication means in response to the processing means receiving a transmission signal from the communication means.

The data storage includes a template storage area for storing the medication schedule, the temperature schedule, the breathing template and other templates that are personalised to the user. The storage and transmission of data will be consistent with industry standard security protocols, such as the use of encryption algorithms for secure storage and transmission.

The external casing includes a protrusion and a confronting zone disposed a fixed distance apart for the purposes of targeting temperature taking.

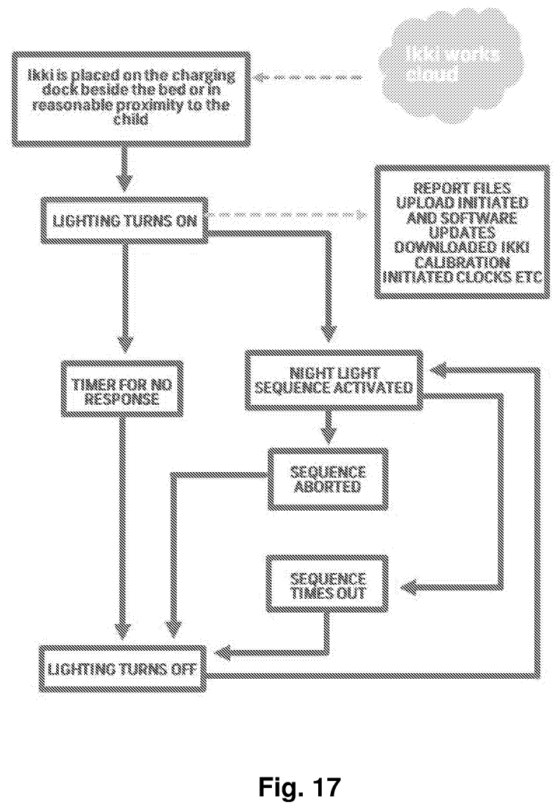

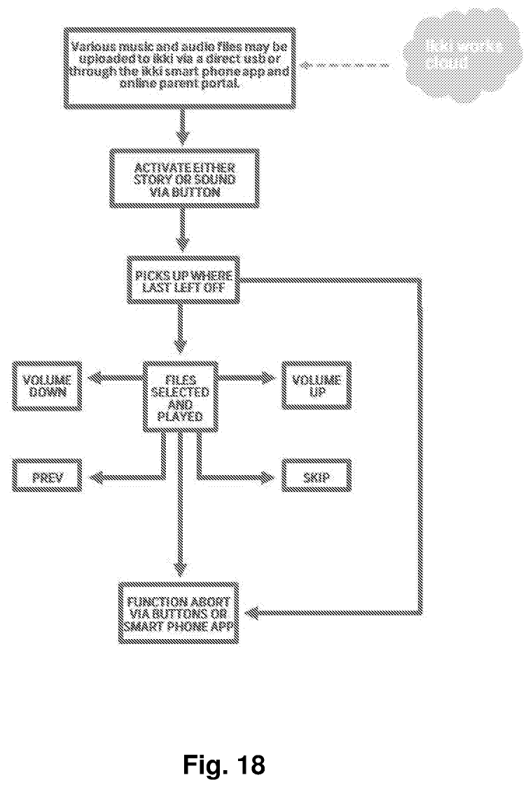

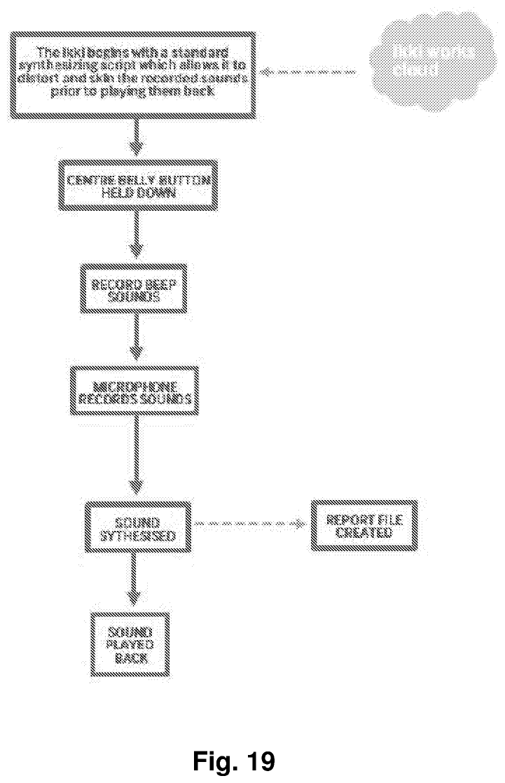

The drawings show flowcharts that describe how other processes and functions are implemented. These include a night light function shown in FIG. 17, a sounds and stories function shown in FIG. 18, a babble speak function shown in FIG. 19 and a wake up sequence shown in FIG. 21

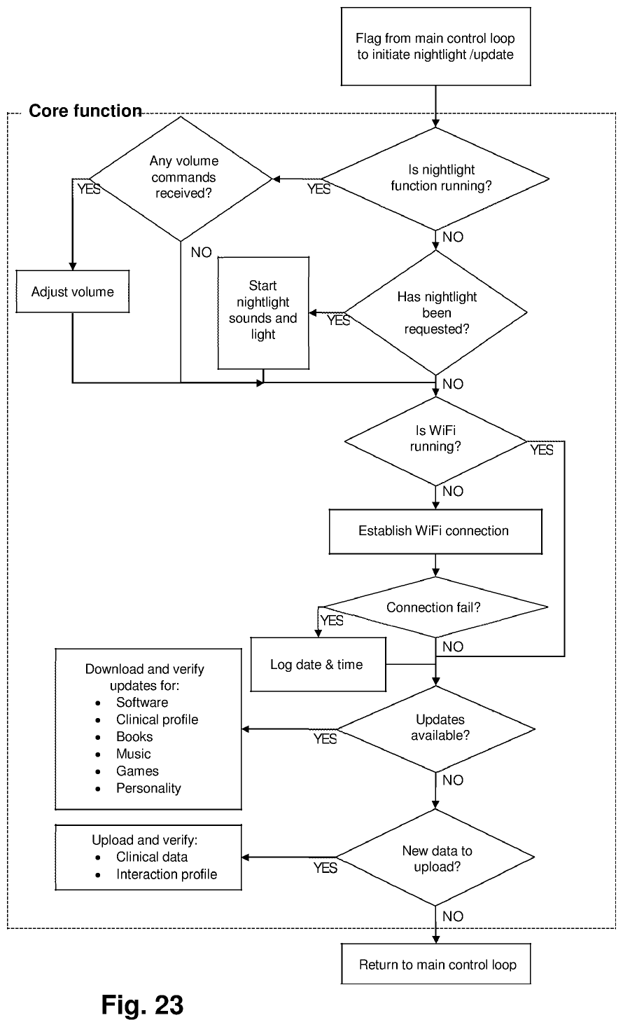

The night light function is an important function performed by the companion robot as it is when the bulk of data transfer occurs, typically via industry standard encryption. This is also when the software is able to update according to artificial intelligence to enable the companion robot to learn to adapt and evolve according to the characteristics and personality of the patient. This adaptation and evolution may include monitoring the nature of the interactions with the patient, such as the games played and proficiency at playing, music listed to, stories listened to, the time of day they complete various interactions, and how active the patient is. The companion robot may then evolve to offer new games, music, stories and activities of a similar type as already conducted by the patient. The update function provided by the night light function is implemented according to the flowchart shown in FIG. 23.

The sounds and stories facility may be enjoyed by the sounds and stories process interacting with the speaker or with a pair of headphones. The features are similar to a traditional personal portable music device with aspects such as volume and selection being facilitated through the buttons presented on the button membrane 33.

The babble speak function provides the user with an audible mirror of terms and phrases spoken to the companion robot 11. The feature is relatively simple and acts as a listen and repeat interaction through which over time the robot will adapt to the voice of the patient and develop a unique tone and character.

The wake up sequence makes for peaceful morning starts and allows for users to initiate a wake up time that suits any treatments or medication schedules that are required.

The overall method adopted by the system for functioning as a companion robot providing services essentially includes:
(i) probing an environment in which the companion robot and user is disposed and generating data in respect of the environment
(ii) sensing information about the user and generating data containing the information;
(iii) communicating sensorially perceptive information to the user and data containing information to a remote processing means; and
(iv) performing prescribed functions with the data in respect of the environment and the information about the user.

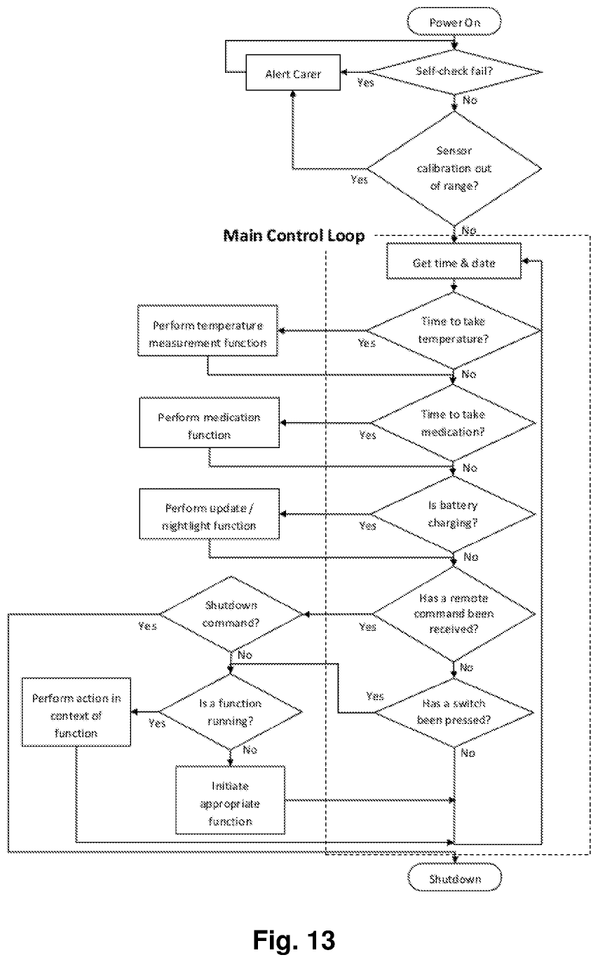

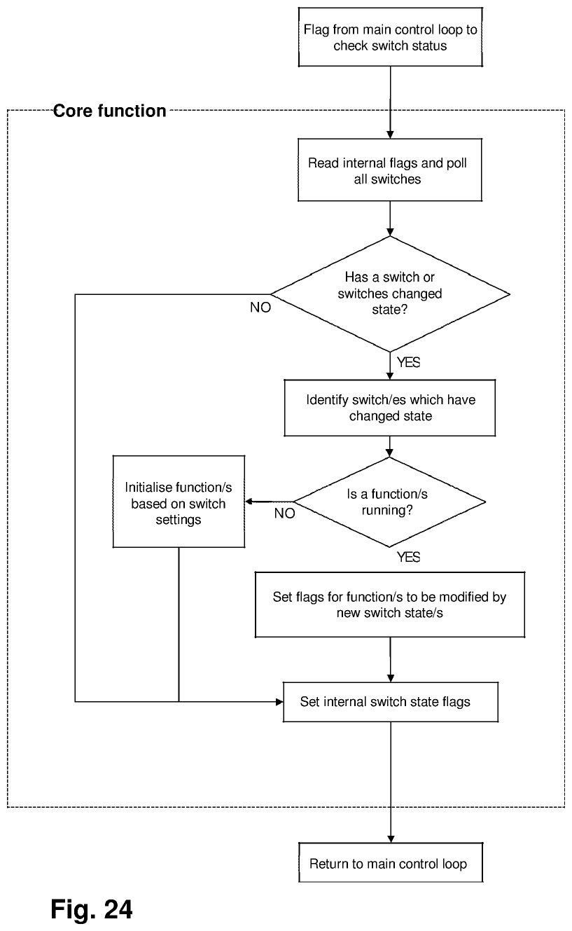

A flowchart showing how the start-up routine and main control loop for implementing the method using the aforementioned system in accordance with the present embodiment is shown in FIG. 13. Further, the flowchart for implementing a switch polling routine as a sub-routine of the main control loop is shown in FIG. 24.

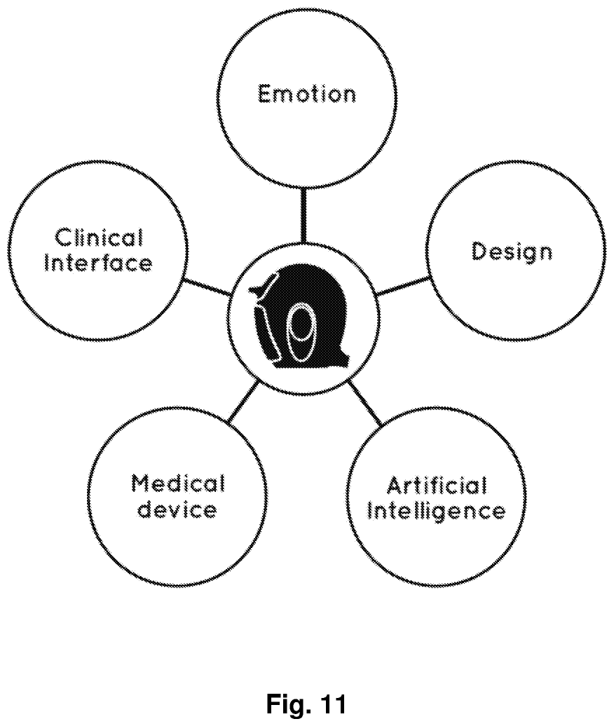

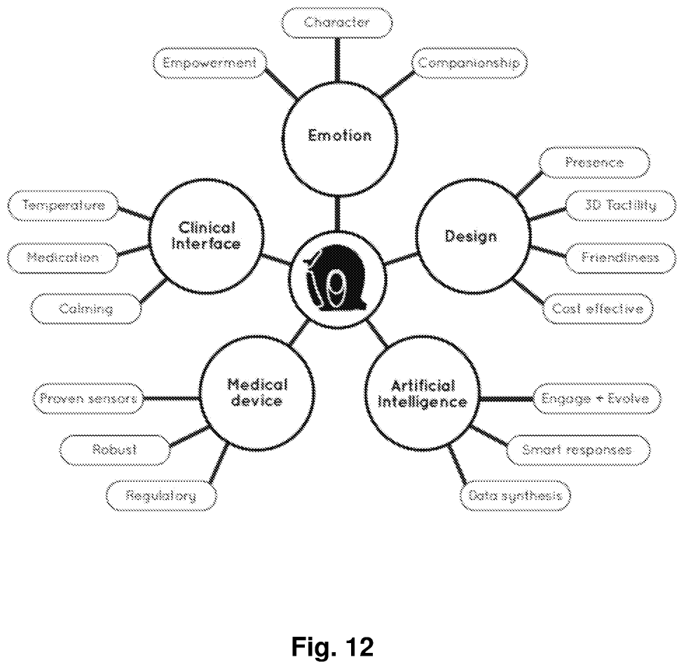

FIG. 11 provides a diagram showing the core principles of the companion robot. These include emotion, design, artificial intelligence, medical device, and clinical interface. The core principles can be customised for each user. FIG. 12 shows one exemplary customisation where emotion includes empowerment, character and companionship; design includes presence, three dimensional tactility, friendliness, and cost effective; artificial intelligence includes engage and evolve, smart responses, data synthesis; medical device includes proven sensors, robust, regulatory; and clinical interface includes temperatures, medication, and calming.

The principle of empowerment is intended to give the patient some responsibility for aspects of their treatment, and the ability to dictate what happens to the companion robot.

The principle of character is provided through the development of characteristics, for example, song, music and game selection, based on the patient's own preferences.

The principle of companionship is provided by the companion robot being robust and portable, going everywhere with the patient, and calming them in moments of stress.

The principle of presence is provided through the physicality of the system, engaging all of the senses at various times.

Three-dimensional tactility provides a rich variety of user interactions and experiences not available on essentially two-dimensional devices such as smartphones and tablets.

Friendliness is provided through endearing audio and visual responses to user input through voice and touch.

Cost effectiveness is achieved through robust design for manufacture and use of standard components.

The concept of engage and evolve is provided through a range of stories, music and games which can evolve, for example new stories and music, or unlocking higher game levels, as the patient's needs change.

Smart responses occur whereby the nature of the response, for example, audio or visual, adapts to the nature of the interactions with the user.

Data synthesis enables generation of accurate, reliable reporting of clinical data such as temperature and medication compliance over time.

The use of sensors with proven technology, including well-developed hardware platforms and robust software libraries, ensures accurate measurement of clinically relevant data.

Robust design, including hardware and software elements, enables the companion robot to accompany the patient everywhere and still perform key roles.

The companion robot and associated documentation will meet the regulatory requirements for a Class 1 medical device with measuring function.

Temperature measurement can be carried out by the patient using a non-contacting infra-red sensor which allows for quick and simple measurement with wireless reporting to carers and the clinical team.

Medication compliance is determined through the use of RFID technology.

The calming function uses a combination of temperature sensing and audio-visual cues to provide an immersive experience which slows the patient's breathing rate to a steady level.

Advantageously, the invention is able to provide a number of key elements that provide significant benefit in the described healthcare application. These include the ability to carry out clinical measurements, empowerment of the patient, robustness, cost effective to allow purchase by a patient, companionship, engagement through complementary personality, games, stories and music, calming, relieves stress for the patient and their family, and provides communication between carers, family, clinicians and patient. Further advantageously, the platform technology is applicable to a wide range of applications.

It should be appreciated that the scope of the present invention is not limited to the specific description of the embodiments and that the described embodiments are only illustrative, with modifications and different combinations of features described constituting new embodiments still forming part of the invention, being consistent with the spirit and scope of the invention.

### Interpretation

Unless specifically stated otherwise, it is appreciated that throughout the specification discussions utilizing terms such as “processing,” “computing,” “calculating,” “determining”, “analysing” or the like, refer to the action and/or processes of a computer or computing system, or similar electronic computing device, that manipulate and/or transform data represented as physical, such as electronic, quantities into other data similarly represented as physical quantities.

In a similar manner, the term “controller” or “processor” may refer to any device or portion of a device that processes electronic data, e.g., from registers and/or memory to transform that electronic data into other electronic data that, e.g., may be stored in registers and/or memory. A “computer” or a “computing machine” or a “computing platform” may include one or more processors.

The methodologies described herein are, in one embodiment, performable by one or more processors that accept computer-readable (also called machine-readable) code containing a set of instructions that when executed by one or more of the processors carry out at least one of the methods described herein. Any processor capable of executing a set of instructions (sequential or otherwise) that specify actions to be taken are included.

Thus, one example is a typical processing system that includes one or more processors. Each processor may include one or more of a CPU, a graphics processing unit, and a programmable DSP unit. The processing system further may include a memory subsystem including main RAM and/or a static RAM, and/or ROM. A bus subsystem may be included for communicating between the components. The processing system further may be a distributed processing system with processors coupled by a network. If the processing system requires a display, such a display may be included, e.g., a liquid crystal display (LCD) or a cathode ray tube (CRT) display. If manual data entry is required, the processing system also includes an input device such as one or more of an alphanumeric input unit such as a keyboard, a pointing control device such as a mouse, and so forth.

The term memory unit as used herein, if clear from the context and unless explicitly stated otherwise, also encompasses a storage system such as a disk drive unit. The processing system in some configurations may include a sound output device, and a network interface device. The memory subsystem thus includes a computer-readable carrier medium that carries computer-readable code (e.g., software) including a set of instructions to cause performing, when executed by one or more processors, one of more of the methods described herein. Note that when the method includes several elements, e.g., several steps, no ordering of such elements is implied, unless specifically stated. The software may reside in the hard disk, or may also reside, completely or at least partially, within the RAM and/or within the processor during execution thereof by the computer system. Thus, the memory and the processor also constitute computer-readable carrier medium carrying computer-readable code.

Reference throughout this specification to “one embodiment”, “some embodiments” or “an embodiment” means that a particular feature, structure or characteristic described in connection with the embodiment is included in at least one embodiment of the present disclosure. Thus, appearances of the phrases “in one embodiment”, “in some embodiments” or “in an embodiment” in various places throughout this specification are not necessarily all referring to the same embodiment. Furthermore, the particular features, structures or characteristics may be combined in any suitable manner, as would be apparent to one of ordinary skill in the art from this disclosure, in one or more embodiments.

In the claims below and the description herein, any one of the terms comprising, comprised of or which comprises is an open term that means including at least the elements/features that follow, but not excluding others. Thus, the term comprising, when used in the claims, should not be interpreted as being limitative to the means or elements or steps listed thereafter. For example, the scope of the expression a device comprising A and B should not be limited to devices consisting only of elements A and B. Any one of the terms including or which includes or that includes as used herein is also an open term that also means including at least the elements/features that follow the term, but not excluding others. Thus, including is synonymous with and means comprising.

It should be appreciated that in the above description of exemplary embodiments of the disclosure, various features of the disclosure are sometimes grouped together in a single embodiment, Fig., or description thereof for the purpose of streamlining the disclosure and aiding in the understanding of one or more of the various inventive aspects. This method of disclosure, however, is not to be interpreted as reflecting an intention that the claims require more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this disclosure.

Furthermore, while some embodiments described herein include some but not other features included in other embodiments, combinations of features of different embodiments are meant to be within the scope of the disclosure, and form different embodiments, as would be understood by those skilled in the art. For example, in the following claims, any of the claimed embodiments can be used in any combination.

In the description provided herein, numerous specific details are set forth. However, it is understood that embodiments of the disclosure may be practiced without these specific details. In other instances, well-known methods, structures and techniques have not been shown in detail in order not to obscure an understanding of this description.

Similarly, it is to be noticed that the term coupled, when used in the claims, should not be interpreted as being limited to direct connections only. The terms “coupled”, “connected,” “attached”, and/or “joined, along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B which may be a path including other devices or means. “Coupled” may mean that two or more elements are either in direct physical, electrical or optical contact, or that two or more elements are not in direct contact with each other but yet still co-operate or interact with each other. In contrast, when a component is referred to as being “directly coupled”, “directly attached”, and/or “directly joined” to another component, there are no intervening elements present.

Various aspects of the present devices, systems, and methods may be illustrated with reference to one or more exemplary embodiments. As used herein, the term “exemplary” means “serving as an example, instance, or illustration,” and should not necessarily be construed as preferred or advantageous over other embodiments disclosed herein.

Thus, while there has been described what are believed to be the preferred embodiments of the disclosure, those skilled in the art will recognize that other and further modifications may be made thereto without departing from the spirit of the disclosure, and it is intended to claim all such changes and modifications as fall within the scope of the disclosure. For example, any formulas given above are merely representative of procedures that may be used. Functionality may be added or deleted from the block diagrams and operations may be interchanged among functional blocks. Steps may be added or deleted to methods described within the scope of the present disclosure.

1. A system for functioning as a companion robot for a user, the system including:
one or more sensors adapted to:
(i) probe an environment in which the companion robot and user is disposed and generate data in respect of the environment; and
(ii) sense information about the user and generate data containing the information;
communication means to communicate:
(i) sensorially perceptive information to the user; and
(ii) data containing information to a remote processing means;
processing means to:
(i) receive data from each sensor;
(ii) process the data to extract information therefrom and perform predetermined functions using the information; and
(iii) provide output information and data to the communication means to communicate; and an external casing at least substantially encapsulating the system.

2. A system according to claim 1, wherein the sensors include one or more of the following:
(i) a distance sensor for determining the distance between the surface of an object in a line of sight and the location of the distance sensor;
(ii) a temperature sensor for determining the ambient temperature around the casing;
(iii) an accelerometer for determining the acceleration of the casing;
(iv) a speedometer for determining the speed of the casing;
(v) an RFID (radio frequency identification) interrogation sensor for detecting and interrogating a remote RFID tag associated with the system when brought into a predetermined proximity to the RFID interrogation sensor; and
(vi) a tactile sensor for determining when the casing is touched in a predetermined manner by an external object.

3. A system according to claim 1, wherein the processing means includes one or more of the following processes to perform one or more of the predetermined functions:
(i) an initialisation process invoked for coupling the system to a user;
(ii) a recharging process invoked for charging a battery resident with the system for delivering locally supplied power to the system;
(iii) an RFID interrogating process invoked by the RFID interrogation sensor for identifying an object bearing the remote RFID tag and performing a prescribed processing function in relation to the object.

4. A system according to claim 1, wherein the communication means includes one or more of the following communication mechanisms:
(i) lighting;
(ii) sound;
(iii) movement; and
(iv) wireless;
wherein each of the communication mechanisms are individually triggerable by a related process of the processing means.

5. A system according to claim 1, wherein the processing means includes a medication compliance process.

6. A system according to claim 5, wherein the medication compliance process includes one or more of the following sub-processes which are performed in respect of an associated medication event:
(i) a medication reminder invoking process;
(ii) a medication verification process;
(iii) a correct medication asserting process; and
(iv) a logging process for logging the medication event.

7. A system according to claim 1, wherein the processing means includes a temperature taking process.

8. A system according to claim 7, wherein the temperature taking process is programmed to perform one or more of the following sub-processes in respect of a temperature taking event:
(i) a temperature reminder invoking process;
(ii) a temperature calibration process;
(iii) a temperature orientation process to direct the user to orient the system into the correct orientation for the temperature measurement;
(iv) a temperature positioning process to direct the user to position the system at the correct distance from the user's skin for optimal measurement accuracy;
(v) a temperature reading process;
(vi) a temperature comparison process;
(vii) a temperature alert process;
(viii) a logging process for logging the temperature taking event.

9. A system according to claim 6 or claim 8, wherein the logging process for logging events includes:
(i) a missing event tracking process for logging missed events;
(ii) an alert function for invoking the communication means to send an alert signal to the remote processing means in accordance with an alert profile customised for the user in respect of events that have been missed for a prescribed number of times; and
(iii) an alert profile interrogating process to derive an alert flag for missing events from the alert profile and trigger the alert function when the missed number of events tracked by the missing event tracking process matches the alert flag.

10. A system according to claim 1, wherein the processing means includes a frustration process that is programmed to perform the following functions in respect of a frustration event:
(i) reading an accelerometer repeatedly once the frustration process has initiated;
(ii) determining if a change of greater than a specified threshold has been detected in the x, y or z axis of movement between two successive readings; and
(iii) if such a change is detected, providing a cue to the user.

11. A system according to claim 1, wherein the processing means includes a reminder process.

12. A system according to claim 11, wherein the reminder process includes:
(i) an audio-visual cue selector to invoke the communication means and generate a corresponding communication mechanism related to the particular type of invoking process triggering the reminder process;
(ii) a time reminder to signal the communication means to assert sensorially perceptible information indicative of the event being reminded in the absence of a response within a prescribed time from the initial reminder;
(iii) a medication reminder or temperature taking reminder to signal the communication means to assert sensorially perceptible information indicative of a medication reminder or temperature taking reminder, as appropriate.

13. A system according to claim 1, wherein the processing means invokes a calming process.

14. A system according to claim 13, wherein the calming process is programmed to perform one or more of the following sub-processes in respect of a calming event:
(i) a light cue process;
(ii) an audio cue process;
(iii) a temperature reading process;
(iv) a high temperature detection process;
(v) a breathing detection process.

15. A system according to claim 1, wherein the processing means invokes a voice sensing and modifying process to sense when a vocal sound is generated by a user, including:
a linguistic detection process; and
a voice responding process for generating a response based on the sensed vocal sound.

16. A system according to claim 15, wherein the voice responding process includes:
(i) use of a microphone to detect vocal sounds;
(ii) conversion of the analogue audio signal generated by the microphone into a digital format;
(iii) use of real-time sound processing software to modify the digital audio signal as it is received;
(iv) use of audio components to play back the modified audio signal.

17. A system according to claim 15, wherein the voice responding process includes analysis of a detected vocal sound, and generation of a response relevant to the result of the analysis.

18. A system according to claim 1, wherein the processing means includes a motion process for interacting with a motion sensor, including:
(i) an accelerometer to measure movement in the x, y and z planes of movement;
(ii) software to analyse the output of the accelerometer and provide an appropriate response.

19. A method for functioning as a companion robot for a user, the method including:
(i) probing an environment in which the companion robot and user is disposed and generating data in respect of the environment;
(ii) sensing information about the user and generating data containing the information;
(iii) communicating sensorially perceptive information to the user and data containing information to a remote processing means; and
performing prescribed functions with the data in respect of the environment and the information about the user.
